(19) United States
(12) Patent Application Publication
(10) Pub. No.: US 2006/0147482 A1
(43) Pub. Date: Jul. 6, 2006

(54) Oral liquid pharmaceutical composition of leukotriene antagonists

(75) Inventor: Chin-Yin Chang, Hsinchu Hsien (TW)

Correspondence Address: THOMAS, KAYDEN, HORSTEMEYER & RISLEY, LLP, 100 GALLERIA PARKWAY, NW, STE 1750, ATLANTA, GA, 30339-5948, US

(73) Assignee: Center Laboratories, Inc.

(21) Appl. No.: 11/223,280

(22) Filed: Sep. 9, 2005

(30) Foreign Application Priority Data
Jan. 4, 2005 (TW) 94100182

(51) Int. Cl. A61K 31/47; A61K 9/00

(52) U.S. Cl. 424/400; 514/311

(57) ABSTRACT

An oral liquid pharmaceutical composition of leukotriene antagonists is described and is used an anti-asthmatic, anti-allergic, anti-inflammatory, and cytoprotective agent. The composition is a buffer solution with the Montelukast or its pharmaceutically acceptable salt. The pH value of the buffer solution is between about 7 and 11. The buffer solution contains water, pharmaceutically acceptable alcohol, buffer agents, and pharmaceutically acceptable additives.

### RELATED APPLICATIONS

The present application is based on, and claims priority from, Taiwan Application Serial Number 94100182, filed Jan. 4, 2005, the disclosure of which is hereby incorporated by reference herein in its entirety.

### BACKGROUND

1. Field of Invention

The present invention relates to an anti-allergic oral liquid pharmaceutical composition. More particularly, the present invention relates to an oral liquid pharmaceutical composition of leukotriene antagonists.

2. Description of Related Art

The number of people suffering from allergy-related diseases such as, for example, hay fever, allergic rhinitis, poison ivy, and asthma has increased in recent years. The species and symptoms of the allergy-related diseases are different and cover a great range. In the case of asthma, there are one hundred and fifty-five million asthma patients around the world. The number of people who die from asthma is about two hundred thousand per year. The number of asthma patients is increasing worldwide at the rate of 20 percent to 50 percent per decade. The cost of treating this kind of chronic diseases is huge. The cost for treating asthma is around sixty billion USD per year in the United States, thirty billion USD in Germany, sixteen billion USD in Britain, and twenty billion NTD in Taiwan. The pharmaceutical market for asthma drugs in these countries is fifty-five billion USD each year. Asthma thus constitutes a higher threat to human health than AIDS or cancer.

Leukotriene is a chemical material created inside human body and it plays an important role in inflammatory reactions. It is released from a stimulated respiratory tract during onset of an asthma attack. Leukotriene combines with receptors on the respiratory tract cells, resulting in some symptoms of asthma such as respiratory tract constriction, edema, and the increase of salivary secretion.

Asthma pharmaceuticals divided into two categories, anti-inflammatory pharmaceuticals and bronchodilators. Anti-inflammatory drugs can alleviate or terminate the asthmatic inflammatory reaction in the respiratory tract and lower the sensitivity of the respiratory tract. Besides, it can be used to prevent the occurrence of a bronchial inflammatory reaction. Bronchodilators are primarily used to relax the smooth muscle of the respiratory tract during an asthmatic inflammatory reaction, which reaction causes the smooth muscle to contract. However, bronchodilators do not treat the inflammatory reaction or lower the sensitivity of the respiratory tract.

Asthma drugs are divided into two categories because asthma is not only respiratory tract contraction but also is a chronic inflammatory reaction. Bronchodilators are necessary to alleviate the asthmatic symptom like respiratory tract contraction, and anti-inflammatory drugs are also necessary to control the inflammatory reaction.

Generally, anti-inflammatory drugs are usually used as preventive medicine. The major anti-inflammatory drugs in use nowadays are steroids, but the most popular new drugs are Leukotriene receptor antagonists.

U.S. Pat. No. 5,565,473 discloses a Leukotriene receptor antagonist having following chemical formula I. The structures of the constituents and related symbols in the following chemical formula I can be found in the content of U.S. Pat. No. 5,565,473. All the related compounds disclosed by U.S. Pat. No. 5,565,473 can be used in this invention.

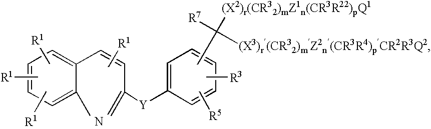

Based on this patent, Merck Sharp & Dohme applied for approval of a kind of leukotriene receptor antagonist, Montelukast, from the Food and Drug Administration. The structure of Montelukast is shown as chemical formula II. A tablet of Montelukast or its salt can efficiently control asthma symptoms after a two-week buildup in the system and thus prevent asthma attacks. For some asthma patients, especially for children below six years old and the elderly, oral tablets are not convenient. The tablet is usually ground into powder before administration to children and the elderly because they have difficulty in swallowing tablets. When the tablet is ground into powder, impurities are introduced during the grinding process and the drug dosage is hard to control.

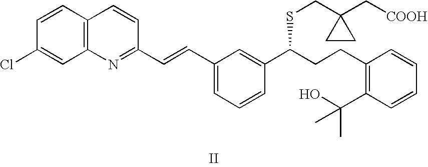

### SUMMARY

Leukotriene receptor antagonists, especially Montelukast or its salt like chemical formula II is hard to dissolve in water. The result of solubility of Montelukast is referred to the following comparison example 1. Generally speaking, the solubility of the compound in the water increases when an organic solvent or an emulsifier is added into the water. The results of the comparison example 2 and 3 illustrate that although Montelukast or its salt can dissolve in water containing ethanol or propylene glycol, they are still very unstable in this kind water solution. Although the water solubility of Montelukast can be increased by adjusting the pH value of the solution illustrated in the comparison example 2 and 3, the concentration of Montelukast or its salt cannot be detected by HPLC after dissolution in water for thirty days.

It is therefore an aspect of the present invention to provide an oral liquid pharmaceutical composition of leukotriene antagonists like chemical formula II which is easily be taken by asthma patients.

It is another an aspect of the present invention to provide an oral liquid pharmaceutical composition of leukotriene antagonists, like chemical formula II, which is very stable.

In accordance with the foregoing and other aspects of the present invention, the present invention provides an oral liquid pharmaceutical composition of leukotriene antagonists like chemical formula II, at least comprising Montelukast or its pharmaceutically acceptable salt, pharmaceutically acceptable alcohol, a buffer agent and a pharmaceutically acceptable additive. The pH value of the buffer solution is 7-11. The preferred pH value of the buffer solution is 8-10. The more preferred pH value of the buffer solution is 8.5-9.5. The amount of Montelukast or its salt in the oral liquid pharmaceutical composition is about 0.01-20% w/v. The pharmaceutically acceptable alcohol can be ethanol or propylene glycol. The amount of the pharmaceutically acceptable alcohol in the oral liquid pharmaceutical composition is about 1-40% (volume ratio). The pharmaceutically acceptable buffer agent is used to form a buffer solution having a pH value of 7-11. The preferred buffer agent is preferably phosphoric acid/hydroxide, phosphate salt/hydroxide, boric acid/potassium chloride/hydroxide, tetraborate/inorganic acid, tetraborate/hydroxide or carbonate/bicabonate. The amount of the buffer agent is about 0.1-20% w/v.

The pharmaceutically acceptable additive can comprise selectively an emulsifier, a sweetener, a preservative, a humectant, an edible pigment or an edible essence. The emulsifier can increase the solubility of Montelukast or its salt in the oral liquid pharmaceutical composition. Among theses additives, the emulsifier can be a natural emulsifier, an anionic emulsifier or a nonionic emulsifier. The anionic emulsifier can be sodium dodecyl sulfate or sodium octadecyl sulfate. The nonionic emulsifier can be sorbitol anhydrate or polyvinyl chloride sorbitol anhydrate. Polyvinyl chloride sorbitol anhydrate can be Tween 80. The amount of the emulsifier is about 0.05-5.0% w/v. The sweetener can be sucrose or Equal artificial sweetener. The Equal artificial sweetener can be saccharin, saccharin sodium, aspartame, sorbitol, manntitol, xylitol or acesulfame potassium. The amount of sweetener is about 0.02-10% w/v. The preservative can be methylparaben, benzoic acid or its salt. The amount of the preservative is less than 0.5% w/v. The preferred amount of the preservative is less than 0.2% w/v. The humectant can be glycerine. The edible pigment can be tatrazine, sunset yellow FCF or cochineal red A, or new coccin. The edible essence can be lemon essence or yogurt essence.

In conclusion, the invention provide a method to dissolve effectively leukotriene antagonists in a water solution to form an oral liquid pharmaceutical composition by using a buffer agent to adjust the pH value of the water solution and to keep the leukotriene antagonists stable therein.

### DESCRIPTION OF THE PREFERRED EMBODIMENTS

The invention is related to an oral liquid pharmaceutical composition of Montelukast or its salt-like chemical formula II. These and other features, aspects, and advantages of the present invention will become better understood with reference to the following embodiments and comparison.

The manufacturing method that the embodiments and comparisons disclose is to dissolve the additives (or the buffer agents) in water and then to adjust the pH value of the solution to the desired range of 7-11 by adding acid or base into it. At first, an appropriate amount of Montelukast or its salt is dissolved in ethanol or propylene glycol. The organic phase is mixed with the water phase. Finally, acid or base is added to the water solution to adjust its pH value to the desired range of 7-11. The preferred pH value of the buffer solution is about 8-10. The more preferred pH value of the buffer solution is about 8.5-9.5. Some essence can selectively be added in the buffer solution.

The method in this invention used to detect the concentration of Montelukast or its salt is HPLC. The mobile phase is the mixture of 0.05 M Ammonium acetate and methanol. The ratio between them is 3:17. The flow speed of the mobile phase in use is 1.5 ml per minute. The injection volume is 20 μl each time. Montelukast or its salt is detected by ultra violet light with a wavelength of 254 nm. The pre-process method of standard solution and sample solution is to dilute them with the mixture of methanol and water. The ratio between methanol and water is 3:7.

### COMPARISON EXAMPLE 1

The object was to form a water solution whose concentration of the sodium salt of Montelukast was 0.2% w/v. At first, suitable amount of the sodium salt of Montelukast was dissolved in hot propylene glycol. White deposition was formed while water was added into propylene glycol. From the result of comparison 1, the solubility of the sodium salt of Montelukast was poor with water.

### COMPARISON EXAMPLE 2

The sodium salt of Montelukast was dissolved in ethanol. Saccharin sodium salt, sodium benzoate, glycerol, an edible pigment and an edible essence were dissolved in water. 1N sodium hydroxide was then added to the water solution to adjust its pH value to the range of 9-10. After mixing the water phase and organic phase, the pH value of the solution was adjusted to the range of 9-10. The sodium salt of Montelukast was completely dissolved when its concentration in solution was 0.2% w/v. After the resultant solution was stored at the temperature of 40° C. for 25 days, the concentration of the sodium salt of Montelukast couldn't be detected by HPLC.

### COMPARISON EXAMPLE 3

The sodium salt of Montelukast was dissolved in propylene glycol. Sucrose, sodium benzoate, glycerol, an edible pigment and an edible essence were dissolved in water. 1N sodium hydroxide was then added to the water solution to adjust its pH value to the range of 9-10. After mixing the water phase and organic phase, the pH value of the solution was adjusted to the range of 9-10. The sodium salt of Montelukast was completely dissolved when its concentration in solution was 0.2% w/v. After the resultant solution was stored at the temperature of 40° C. for 25 days, the concentration of the sodium salt of Montelukast couldn't be detected by HPLC. From the result of comparison 2 and comparison 3, the basic condition enhances the solubility of the sodium salt of Montelukast, but the sodium salt of Montelukast appeared to be unstable in the basic solution.

Comparison 4

The process procedure was the same as for comparison 2. However, cellulose was added to the solution to make a suspension. The cellulose used was hydroxylpropyl methycellulose. The sodium salt was equally distributed therein. After the suspension was stored at a temperature of 40° C. for 6 days, the concentration of the sodium salt of Montelukast in suspension decreased to 66%.

From the results of above three comparisons, the basic condition enhances the solubility of the sodium salt of Montelukast. But the sodium salt of Montelukast appeared to be unstable in the basic solution. The sodium salt of Montelukast was not even stable in a suspension.

### EXAMPLE 1

The sodium salt of Montelukast was dissolved in propylene glycol. Dipotassium orthophosphate, potassium dihydrogen phosphate, saccharin sodium salt, sodium benzoate, an edible pigment and an edible essence were dissolved in water. 1N sodium hydroxide was then added to the water solution to adjust its pH value to the range of pH 9-10. After mixing the water phase and organic phase, the pH value of the solution was adjusted to the range of pH 9-10 by using the 1N sodium hydroxide. The sodium salt of Montelukast was completely dissolved when its concentration in solution was 0.2% w/v. The resultant solution was tested for its stability at temperatures of 40° C. and 60° C., separately. After the resultant solution was stored at the temperature of 40° C. for 150 days, the concentration of the sodium salt of Montelukast was unchanged. After the resultant solution was stored at a temperature of 60° C. for 120 days, the concentration of the sodium salt of Montelukast was unchanged.

The concentration of the phosphorate in water solution was about 0.5-7% w/v. In this embodiment, the concentration of the phosphorate in water solution was about 1-2% w/v.

### EXAMPLE 2

The sodium salt of Montelukast was dissolved in propylene glycol. Dipotassium orthophosphate, potassium dihydrogen phosphate, saccharin sodium salt, sodium benzoate, tween 80, an edible pigment and an edible essence were dissolved in water. 1N sodium hydroxide was then added to the water solution to adjust its pH value to the range of pH 9-10. After mixing the water phase and organic phase, the pH value of the solution was adjusted to the range of pH 9-10 by using the 1N sodium hydroxide. The sodium salt of Montelukast was completely dissolved when its concentration in solution was 0.2% w/v. The resultant solution was tested for stability at temperatures of 40° C. and 60° C., separately. After the resultant solution was stored at a temperature of 40° C. for 150 days, the concentration of the sodium salt of Montelukast was unchanged. After the resultant solution was stored at a temperature of 60° C. for 120 days, the concentration of the sodium salt of Montelukast was unchanged.

The concentration of the phosphorate in water solution was about 0.5-7% w/v. In this embodiment, the concentration of the phosphorate in water solution was about 1-2% w/v. The amount of tween 80 in the solution was 0.075% w/v.

### EXAMPLE 3

The sodium salt of Montelukast was dissolved in propylene glycol. Boric acid, potassium chloride, saccharin sodium salt, sodium benzoate, an edible pigment and an edible essence were dissolved in water. 1N sodium hydroxide was added to the water solution to adjust its pH value to the range of pH 9-10. After mixing the water phase and organic phase, the pH value of the solution was adjusted to the range of pH 9-10 by using the 1N sodium hydroxide. The sodium salt of Montelukast was completely dissolved when its concentration in solution was 0.1% w/v. The resultant solution was tested for its stability at temperatures of 40° C. and 60° C., separately. After the resultant solution was stored at a temperature of 40° C. for 45 days, the concentration of the sodium salt of Montelukast was unchanged. After the resultant solution was stored at a temperature of 60° C. for 45 days, the concentration of the sodium salt of Montelukast was unchanged.

The concentration of the borate in water solution was about 0.1-3% w/v. In this embodiment, the concentration of the borate in water solution was about 0.31% w/v. In this embodiment, the amount of potassium chloride in the solution was 0.37% w/v.

### EXAMPLE 4

The sodium salt of Montelukast was dissolved in propylene glycol. Dipotassium orthophosphate, saccharin sodium salt, sodium benzoate, an edible pigment and an edible essence were dissolved in water. 1N sodium hydroxide were added to the water solution to adjust its pH value to the range of pH 9-10. After mixing the water phase and organic phase, the pH value of the solution was adjusted to the range of pH 9-10 by using the 1N sodium hydroxide. The sodium salt of Montelukast was completely dissolved when its concentration in solution was 0.1% w/v. After the resultant solution was stored at a temperature of 40° C. for 210 days, the concentration of the sodium salt of Montelukast was unchanged.

The concentration of the phosphorate in water solution was about 0.1% w/v-3% w/v. In this embodiment, the concentration of dipotassium orthophosphate in water solution was about 0.31% w/v.

### EXAMPLE 5

The sodium salt of Montelukast was dissolved in propylene glycol. Dipotassium orthophosphate, sodium dihydrogen phosphate, saccharin sodium salt, sodium benzoate, an edible pigment and an edible essence were dissolved in water. 1N sodium hydroxide was added to the water solution to adjust its pH value to the range of pH 9-10. After mixing the water phase and organic phase, the pH value of the solution was adjusted to the range of pH 9-10 by using the 1N sodium hydroxide. The sodium salt of Montelukast was completely dissolved when its concentration in solution was 0.2% w/v. The resultant solution was tested for its stability at temperatures of 40° C. and 60° C., separately. After the resultant solution was stored at a temperature of 40° C. for 150 days, the concentration of the sodium salt of Montelukast was unchanged. After the resultant solution was stored at a temperature of 60° C. for 120 days, the concentration of the sodium salt of Montelukast was unchanged.

The concentration of the phosphorate in water solution was about 0.1-3% w/v. In this embodiment, the concentration of the phosphorate in water solution was about 1.05% w/v.

### EXAMPLE 6

The sodium salt of Montelukast was dissolved in propylene glycol. Sodium tetraborate, saccharin sodium salt, sodium benzoate, an edible pigment and an edible essence were dissolved in water. 1N hydrochloric acid was added to the water solution to adjust its pH value to the range of pH 9-10. After mixing the water phase and organic phase, the pH value of the solution was adjusted to the range of pH 9-10 by using the 1N hydrochloric acid. The sodium salt of Montelukast was completely dissolved when its concentration in solution was 0.1% w/v. The resultant solution was tested for its stability at temperatures of 40° C. and 60° C. separately. After the resultant solution was stored at a temperature of 40° C. for 45 days, the concentration of the sodium salt of Montelukast was unchanged. After the resultant solution was stored at a temperature of 60° C. for 45 days, the concentration of the sodium salt of Montelukast was unchanged.

The concentration of the tetraborate in water solution was about 0.1-3% w/v. In this embodiment, the concentration of the tetraborate in the solution was 0.38% w/v.

### EXAMPLE 7

Oral liquid pharmaceutical compositions provided in example 1 and examples 2 were done with dissolution test at different pH values such as pH 1.6, pH 4.8 and pH 7.6. After a period time of 120 minutes to 180 minutes, the dissolution curve of the sodium salt of Montelukast of example 1 was about 35-40% and the dissolution curve of the sodium salt of Montelukast of example 2 was about 70-90%.

From the above results of examples and comparisons, basic water solution indeed can enhance the solubility of the sodium salt of Montelukast with the solution but the sodium salt of Montelukast isn't stable therein. When the sodium salt of Montelukast is added to the water solution with the buffer agent, the sodium salt of Montelukast can be stable in the basic water solution. The emulsifier can increase the degree of dissolution of Montelukast or its salt in different acidic or basic environments.

Utilizing the buffer water solution of pH 7-11 of the invention disclosure with alcohols can effectively form water solution in which the concentration the sodium salt of Montelukast is 0.01-2% w/v. The sodium salt of Montelukast is very stable in the water solution. Added edible essences and sweetners to the solution can form a fragrant and sweet oral liquid pharmaceutical composition. The oral liquid pharmaceutical composition can precisely control the amount of the sodium salt of Montelukast delivered to a human body. It prevents the introduction of an impurity during a grinding process and is easily dissolved for absorption into a human body.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An oral liquid pharmaceutical composition of leukotriene antagonists, at least comprising:
a buffer solution having a pH value of between about 7 and about 11, comprising:
water;
pharmaceutically acceptable alcohol;
a buffer agent; and
a pharmaceutically acceptable additive; and
Montelukast or a pharmaceutically acceptable salt thereof, dissolved in the buffer solution.

2. The oral liquid pharmaceutical composition of leukotriene antagonists of claim 1, wherein a pH value thereof is between about 8 and about 10.

3. The oral liquid pharmaceutical composition of leukotriene antagonists of claim 1, wherein a pH value thereof is between about 8.5 and about 9.5.

4. The oral liquid pharmaceutical composition of leukotriene antagonists of claim 1, wherein an amount of the buffer agent is between about 0.1% w/v and about 20% w/v.

5. The oral liquid pharmaceutical composition of leukotriene antagonists of claim 1, wherein the buffer agent is phosphoric acid/hydroxide, phosphate salt/hydroxide, boric acid/potassium chloride/hydroxide, tetraborate/inorganic acid, tetraborate/hydroxide or carbonate/bicabonate.

6. The oral liquid pharmaceutical composition of leukotriene antagonists of claim 5, wherein the buffer agent is preferably phosphoric acid/hydroxide, phosphate salt/hydroxide.

7. The oral liquid pharmaceutical composition of leukotriene antagonists of claim 6, wherein a concentration of the phosphorate is between about 0.5% w/v and about 7% w/v.

8. The oral liquid pharmaceutical composition of leukotriene antagonists of claim 7, wherein a concentration of the phosphorate is between about 1% w/v and about 2% w/v.

9. The oral liquid pharmaceutical composition of leukotriene antagonists of claim 6, wherein a concentration of the borate is between about 0.1% w/v and about 3% w/v.

10. The oral liquid pharmaceutical composition of leukotriene antagonists of claim 1, wherein the pharmaceutically acceptable alcohol is ethanol or propylene glycol.

11. The oral liquid pharmaceutical composition of leukotriene antagonists of claim 1, wherein the pharmaceutically acceptable additive is an emulsifier, a sweetener, a preservative, a humectant, an edible pigment or an edible essence.

12. The oral liquid pharmaceutical composition of leukotriene antagonists of claim 11, wherein the sweetener is sucrose or Equal artificial sweetener.

13. The oral liquid pharmaceutical composition of leukotriene antagonists of claim 11, wherein an amount of sweetener is between about 0.02% w/v and about 10% w/v.

14. The oral liquid pharmaceutical composition of leukotriene antagonists of claim 12, wherein the Equal artificial sweetener is saccharin, saccharin sodium, aspartame, sorbitol, manntitol, xylitol or acesulfame potassium.

15. The oral liquid pharmaceutical composition of leukotriene antagonists of claim 11, wherein the preservative is methylparaben, benzoic acid or its salt.

16. The oral liquid pharmaceutical composition of leukotriene antagonists of claim 11, wherein an amount of the preservative is less than 0.5% w/v.

17. The oral liquid pharmaceutical composition of leukotriene antagonists of claim 11, wherein an amount of the preservative is less than 0.2% w/v.

18. The oral liquid pharmaceutical composition of leukotriene antagonists of claim 11, wherein the humectant is glycerine.

19. The oral liquid pharmaceutical composition of leukotriene antagonists of claim 11, wherein the edible pigment is tatrazine, sunset yellow FCF or cochineal red A, new coccin.

20. The oral liquid pharmaceutical composition of leukotriene antagonists of claim 11, wherein the edible essence is lemon essence or yogurt essence.

21. The oral liquid pharmaceutical composition of leukotriene antagonists of claim 11, wherein the emulsifier is a natural emulsifier, an anionic emulsifier or a nonionic emulsifier.

22. The oral liquid pharmaceutical composition of leukotriene antagonists of claim 11, wherein the anionic emulsifier is sodium dodecyl sulfate or sodium octadecyl sulfate.

23. The oral liquid pharmaceutical composition of leukotriene antagonists of claim 11, wherein the nonionic emulsifier is sorbitol anhydrate or polyvinyl chloride sorbitol anhydrate.
